(19) United States
(12) Patent Application Publication
(10) Pub. No.: US 2012/0259655 A1
(43) Pub. Date: Oct. 11, 2012

(54) Drug Inventory System

(76) Inventor: Steven A. Madreperla, Summit, NJ (US)

(21) Appl. No.: 13/440,649

(22) Filed: Apr. 5, 2012

Related U.S. Application Data
(60) Provisional application No. 61/472,517, filed on Apr. 6, 2011.

(51) Int. Cl. G06Q 50/22; G06Q 10/08

(52) U.S. Cl. 705/2

(57) ABSTRACT

A system and method for managing drug dose inventory for a medical practice includes a server in electronic communication with a database and an interface with practice management software. The system allows users in a medical practice to track drug dose orders, receipt, and the administration of drug doses to patients, and billing for the doses, with a user computer that accesses the server over the Internet.

### CROSS-REFERENCE TO RELATED APPLICATIONS

This patent application claims the benefit, under 35 U.S.C. §119(e), of U.S. Provisional Patent Application Ser. No. 61/472,517, filed on Apr. 6, 2011, the content of which is incorporated herein by reference in its entirety.

### FIELD OF THE INVENTION

The present teachings relate generally to methods and systems for inventory management and, more particularly, to software for managing drug inventory in physician's offices.

### BACKGROUND OF THE INVENTION

Typically, medical drugs are prescribed to patients by medical doctors who then fulfill prescriptions at a pharmacy. In emergencies, drugs may be dispensed directly to patients such as during emergency treatment at hospitals. Some doctors also deliver drugs directly to patients from their office. These drugs often require a doctor to administer the drug such as by injection. When this is done, the doctor may pay the drug company for the drug and then bill the patient's insurance carrier for reimbursement. The doctor may earn a profit that is a fraction of the total drug cost. In some cases, drug doses may be provided by a third party pharmacy (e.g., specialty pharmacy), specified by the patient's insurer, for administration by the physician. The physician may not bill the insurer in these cases but may communicate with the specialty pharmacy to schedule delivery of doses for scheduled patients in a timely manner.

One medical field that has recently begun dispensing drugs in this way is the field of retina disorder treatment. Retinal doctors may deliver several different drugs to patients in their office. For example, Lucentis is one commonly dispensed drug and costs thousands of dollars per injection. An average retina specialist may inject up to ten doses of Lucentis every day. Lucentis is an anti-angiogenic drug that has been approved to treat the “wet” type of age-related macular degeneration (ARMD), a common form of age-related vision loss. Lucentis is injected intravitreally (into the vitreous humour of the eye). Lucentis is expensive and may be injected into a patient frequently, as often as once a month.

Physician's offices lack the ability to accurately track the administration of drugs such as Lucentis, much less the ability to automate inventory control and insurance reimbursement. Therefore, it would be beneficial to have a superior drug inventory system and method of use.

### SUMMARY OF THE INVENTION

The needs set forth herein as well as further and other needs and advantages are addressed by the present embodiments, which illustrate solutions and advantages described below.

The system of the present embodiment includes, but is not limited to, a database storing drug dosage inventory information, a server computer in electronic communication with the database, an interface that receives, over a network, patient information relating to a patient from a physician's office practice management system and stores it in the database, a bar code printer and bar code scanner which are activated and/or read by the system, an inventory controller that tracks the inventory of drug doses for a medical practice, and a display that provides system access to a user computer over a network, including the association of a drug dose with the patient information in the database. The inventory controller determines an inventory mismatch between the physical inventory of drug doses and an inventory count in the database. Additional functionality tracks and facilitates ordering doses for patients requiring specialty pharmacy medications.

The method of the present embodiment includes the steps of, but is not limited to, providing a database storing drug dosage inventory information, providing a server computer in electronic communication with the database, receiving, over a network, patient information relating to a patient from a physician's office practice management system and storing it in the database, tracking, on a computer, the inventory of drug doses for a medical practice, using a barcode printer and scanner, and providing system access to a user computer over a network, including the association of a drug dose with the patient information in the database. The inventory controller determines an inventory mismatch between the physical inventory of drug doses and an inventory count in the database.

Other embodiments of the system and method are described in detail below and are also part of the present teachings.

For a better understanding of the present embodiments, together with other and further aspects thereof, reference is made to the accompanying drawings and detailed description, and its scope will be pointed out in the appended claims.

### BRIEF DESCRIPTION OF THE DRAWINGS

FIG. 1 is a schematic diagram depicting one embodiment of the system according to the present teachings;

FIG. 2 is a schematic diagram depicting the server computer comprising multiple computers; and

FIG. 3 is a schematic diagram depicting data flow of the system of FIG. 1.

### DETAILED DESCRIPTION OF THE INVENTION

The present teachings are described more fully hereinafter with reference to the accompanying drawings, in which the present embodiments are shown. The following description is presented for illustrative purposes only and the present teachings should not be limited to these embodiments. Any computer configuration and architecture satisfying the speed and interface requirements herein described may be suitable for implementing the system and method of the present embodiments

In one embodiment, the system according to the present teachings may track drugs ordered, received, and dispensed by a physician's office (e.g., a medical practice, etc.). The system may interface with the physician's office practice management system (PMS) to determine proper billing and payment, including insurance information, for drugs delivered to patients in the physician's office. Physician's offices typically use a PMS system to manage traditional accounting (e.g., accounts payable, etc.) for the medical practice. Therefore, the system may interface with the PMS for information exchange such as drug dose charges and insurance reimbursement information, although not limited thereto. The system provides functionality that is not performed by the PMS, including real-time drug dosage tracking, dosage inventory management, and profitability analysis, although not limited thereto. As discussed further below, the system provides a number of benefits to assist with the management of drug doses in a physician's office.

The system may provide reporting capability including a number of reports that may be made available to a user. For example, reports may show handling of inventory, billing, and payment information. The system may further provide alerts to predetermined recipients (e.g., practice managers, etc.) by a variety of different communication mediums (e.g., texts, phone calls, faxes, emails, etc.). Alerts may assist in ensuring adequate stocks of drugs are achieved and inform recipients of any billing or inventory irregularities that require investigation. It is to be appreciated that alerts may be specified for any number of events and for any number of different recipients and the present teachings are not limited to any particular embodiment disclosed herein. The system may also warn the user if a drug dose is about to be delivered to a patient whose insurance company may not reimburse the doctor because of the patient's particular diagnosis. The user may also identify particular patients to whom the practice does not wish to deliver a particular drug type (e.g., for reasons of unlikely reimbursement, allergy, etc).

The system according to the present teachings is referred to generically herein as the drug inventory system. While the current system is discussed in connection with retina specialists, it is not to be limited to this particular exemplary embodiment. In fact, any organization that stores and dispenses inventory may utilize the present teachings, including any other medical specialty that dispenses drug doses which they purchase and for which they bill the recipient or a third party. It is to be appreciated that drug doses may comprise pre-measured doses for immediate administration to a patient or may need to be measured from a larger amount (e.g., multi-dose container, etc.), although not limited thereto.

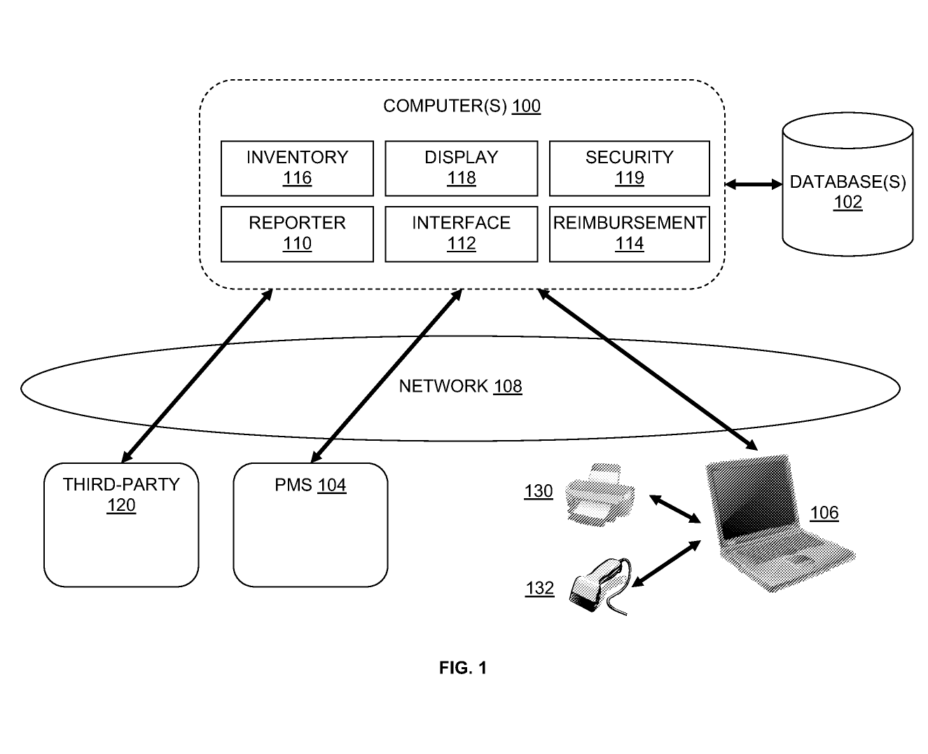

Referring now to FIG. 1, shown is a schematic diagram depicting one embodiment of the system according to the present teachings. The system may comprise a server computer 100 (or computers) in communication with a database 102 (or databases). Exemplary database systems include Microsoft Access, Microsoft SQL Server, MySQL, Oracle, etc., but one skilled in the art would appreciate that any type of data storage system may be utilized with the present teachings. The database 102 may be accessed by the server computer 100 and/or user computer 106 (or computers), although not limited thereto, over a network 108 such as the Internet. Communication in the system may be performed securely using encryption technologies, although not limited thereto.

In one embodiment, the server computer 100 may provide a number of functions to a user computer 106. It is to be appreciated that in a distributed network environment the user computer 106 may access the functions on the server computer 100 over a network 108 such as the Internet. It is specifically appreciated that the system according to the present teachings may be implemented in a client/server architecture whereby the server computer 100 may “serve” functionality to user computer 106. For example, the functionality of the present system may be hosted at a remote location by the server computer 100 and accessed by the user computer 106 through the world wide web with, for example, an Internet browser or related technology (e.g., including terminal services, etc.). However, the present teachings are not limited to any particular embodiment disclosed herein and one skilled in the art would appreciate that the present system may be implemented in any number of different ways, including various network topographies using any number of technologies to provide the disclosed and/or related functionality.

Functionality may reside on the server computer 100, which will be discussed in more detail below. Functionality may include inventory controller 116, display 118, security controller 119, reporter 110, interface 112, and reimbursement controller 114, although not limited thereto. These functional modules may provide for functionality including, although not limited thereto, database entry, drug ordering, order receipt, inventory management, dose transfers, drug dose assignment, payment to drug company (or credit card company, etc.), auditing of drug dose handling, and reporting. These functional modules may be implemented in software executing on computer readable media, although not limited thereto. For example, in one embodiment the inventory controller 116 may comprise inventory software executing on a computer readable medium. Functionality may also be implemented in hardware or a combination of hardware and software, as one skilled in the art would appreciate.

The database 102 (or databases) may provide for centralized access to information used by the system. For example, although not limited thereto, drug types, drug codes, alternate drug information, insurance carrier information, alerts recipients, payment information, appointment information, payment account information, security privileges and security credentials (e.g., username, passwords, etc.) may be stored in the database 102.

PMS 104 (e.g., physician's office practice management system) may have data relating to the management of the physician's office, including accounting, billing, and patient information, although not limited thereto. The user computer 106 may communicate with the PMS 104 over a network 108 such as the Internet. In one embodiment, the system may obtain information from the PMS 104 for storage in the database 102. As an example, the system may obtain dosage billing information from the PMS 104. Using secure communication (e.g., encryption, etc.), for example, the PMS 104 may securely transmit confidential information to the system. In another embodiment, the system functionality may be provided as an add-on (e.g., add-on module, etc.) for the PMS 104 and may be local to the PMS 104. Dosage billing information may include information regarding patients billed with certain billing codes and may include, although not limited thereto, patient information (e.g., name, address, biographical information, medical history, etc.), physician's office information (e.g., physician names, offices/locations, etc.), date of service, date of billing, code billed, units billed, total charge, and insurance information (e.g., company information, plan information, whether the dosage is allowable, allowable amounts, etc.).

The system may obtain information from the PMS 104 for each patient with an upcoming appointment. Having updated patient information in the system allows for assigning dispensed drug doses, discussed further below. Additional information obtained from the PMS 104 may include practice data for each physician in the practice, practice location information (e.g., multiple locations, etc.), appointment information, and insurance information such as plan information, although not limited thereto. It is intended that the information exchange with the PMS 104 allows for the pushing and pulling of any information, whether batched or incremental, and one skilled in the art would appreciate that the present teachings are not limited to any particular embodiment disclosed herein. This communication may be performed by interface 112 over a network 108 such as the Internet.

The database 102 may store drug type information, which may include information about the drugs that the physician's office purchases, delivers to patients and desires to track using the system. In one embodiment, a user (e.g., system administrator, etc.) may initially load the list of drugs and related drug codes into the system from a separate master portal, although not limited thereto. A system user may keep this and other stored information updated as necessary. Drug codes include Healthcare Common Procedure Coding System (HCPCS) codes which are the codes used by Medicare and monitored by the Centers for Medicare and Medicaid Services (CMS) assigned to every task and service a medical practitioner may provide to a Medicare patient. National Drug Code (NDC) numbers, maintained by the FDA and specific to every controlled drug in the country, may also be used. These codes may be used in the search for dose billing data, discussed further below.

Drug information may further include dosage information such as the minimum length of time between injections. Using this information, the system may alert a predetermined recipient (e.g., user, patient, by email, etc.) if a drug is to be assigned to a patient who has received the drug previously within the minimum allowable time. The system may also warn the user if a dose is to be assigned to a patient whose insurance company may not reimburse the practice because of the patient's diagnosis, or if the practice has listed the patient as not to receive the specific drug.

The database 102 may also store alternate drug code information. Occasionally, a particular insurance carrier(s) may decide to use a non-standard drug code or a code that differs from the CMS code. In order to determine if billing information is correct, the system may check to determine if alternative codes rather than standard codes were used when a bill was created in the PMS 104. Alternatively, if there is a two-way interface in place, the system may insert the correct billing code and units directly into the PMS 104.

The database 102 may also store insurance carrier information. In most medical billing situations, there is an amount that is agreed-upon that each insurance company will pay for a particular HCPCS code (e.g., “allowable amount”). A practice may load these amounts in its PMS 104 to determine if it has been paid the correct amount. In one embodiment, the system according to the present teachings may interact with the PMS 104 to obtain this information. If there is no insurance plan identified with a patient, then the allowable amount may be set to the full charge of the drug dose. Similarly, the system may store default allowable amounts.

In one embodiment, the system may communicate with a third party 120 who is an insurance company, although not limited thereto. This may be preferable in situations in which a patient has been administered a drug dose and reimbursement controller 114 may then bill the patient's insurance carrier for reimbursement. This may be accomplished by automatically transmitting the reimbursement request to the insurance company, although not limited thereto.

The database 102 may also store information on alerts. This may include both recipient information as well as the situations in which an alert is to be sent. In this way, the system may alert recipients by popup, sound, text, facsimile, email, phone call, or any other method of alert, upon a certain event or events. For example, a user may provide the name and the email address of recipients who are to be alerted when a certain drug inventory falls below a predetermined level, along with a recommended re-order amount. A pending order may then be created by the system. Alerts may be provided on any number of different events and the present teachings are not limited to any particular embodiment disclosed herein. Alerts may be performed by the reporter 110, discussed further below, although not limited thereto.

In one embodiment, users may define for each office location the number of doses for each drug in inventory, such that when the inventory reaches that number they wish to be alerted to order more. Alerts may also be provided upon notice that the inventory count does not match the calculated number. Alerts may also be generated if billing accuracy for a particular office shows an inaccurate bill in the system for some predetermined period of time (e.g., more than 48 hours, etc., discussed further below), if dose billing data shows a drug dose delivered for which there is no drug delivery assigned, if an inventory count mismatch has occurred, or if a scheduled order was not received within a predetermined amount of time (e.g., within 7 days of order, etc.), although not limited thereto.

The database 102 may also store payment information. In one embodiment, payment information includes credit card information that may be used for purchasing drugs (e.g., from drug companies, distributors, etc.). Drugs will often be ordered and paid for with a credit card that belongs either to the practice or an individual doctor. Credit card information may include information such as name, card number, expiration date, CID number, etc. However, payment information may also include purchase order information, checking account information, or any number of other ways to pay, and the present teachings are not limited to any particular embodiment. The database 102 may also store information for a third party 120 drug vendor with whom the system communicates. This may include, for example, although not limited thereto, drug type, vendor contact information (name, address, phone, website, etc.), payment information, etc.

The system may provide the ability to control access for users using a permissions/privileges/rights scheme and this information may also be stored in the database 102, although not limited thereto. For example, a category of users referred to as “super users” may be defined by the system administrator, giving them access to all functionality, including sensitive areas such as payment information, order entry, and inventory management. It is to be appreciated that each piece of functionality disclosed herein may have a number of different security options for each user or group of users (e.g., physicians, administrative staff, managers, etc.) such as no access, read access, edit access, and add access (e.g., add new information), although not limited thereto. One skilled in the art would appreciate how access may be controlled using permissions and that a system administrator may assign permissions to each user, along with log in credentials (e.g., username, password, etc.).

As shown in FIG. 1, display 118 (e.g., Graphical User Interface or GUI, etc.) may provide for the ability of a user to access the system from a user computer 106 or some other device (e.g., mobile device, phone, tablet, pc, etc.). As discussed above, the display 118 may be provided over a network 108 through the world wide web. In this way, the system may be hosted centrally and be accessed by a user with an Internet browser from any location using log in credentials. The display 118 may provide the necessary screens to access all of the functionality in the system. For example, there may be screens for entering data, administering inventory, running reports, etc.

In one embodiment, the display 118 may provide for the ability of a user to enter drug orders. This may include when doses have been dispensed or are damaged and need to be replaced, although not limited thereto. Information on available drugs (and available vendors) may be stored in the database 102 and the user may select the drug to be ordered as well as the vendor. The user may also provide an invoice number (if available), delivery information (e.g., office delivery location, etc.), drug quantity, and payment information, although not limited thereto. Payment information may include, for example, credit card, purchase order or check number, although not limited thereto. The system may also track the current total dollars assigned to each user (e.g., physician, etc.) for the current calendar year for budgeting purposes. Based upon the vendor contact information, the order may be automatically transmitted by the system via email, facsimile, etc., to a third party 120 vendor. In the alternative, the user may print up an order transmittal (e.g., form, letter, etc.) for mailing to the vendor. The system may provide a library of standard and user-created forms (e.g., invoices, purchase orders, letters, etc.) for users of the system. In one embodiment, the system may pre-load data from the database 102 into the forms to facilitate form creation, although not limited thereto.

The system may use invoices to help track the dispersal and receipt of drug doses. Invoice groups of individual invoices may be “linked” together (e.g., associated with each other in the system) to aid in determining reimbursement. For instance, if a replacement for a damaged dose is returned to the practice by a vendor with a separate invoice number, then that invoice may be linked to the original invoice from which the damaged dose was a part for the purposes of reporting.

Once a user submits an order, the system may track the order. For example, when an order is made to replace damaged doses, it may be useful to assign the new order to an invoice and update the inventory to accommodate for the damaged doses. In this way, the doses may be associated with the invoice for reporting purposes (e.g., profit report, discussed further below). In another embodiment, it may be preferable to track the replacement of doses used for non-paying patients. In this case, the system may track from which invoice the doses were removed for reporting purposes (e.g., this may affect profit report, discussed further below).

When a dose is used to treat a non-paying patient and is eligible for replacement by the vendor, or when a drug dose is removed as damaged and is eligible for replacement by the vendor, the system may track and display such doses for the user to facilitate such doses being properly ordered. And when such doses are received, the system may direct the user as to proper handling and placement in inventory.

A dashboard may be displayed that provides the user with pertinent information about many aspects of the inventory cycle, including pending orders, open orders (to be received), patients with appointments requiring name-specific doses (e.g., specialty pharmacy), doses assigned on the current day, replacement doses that must be ordered, and functionality to match new patients with recently assigned patient identification numbers.

The system may also track drug order receipts and the display 118 may provide the ability to enter information about received drug orders. This may be useful in order to confirm the correct receipt of drugs that were previously ordered. The user may be provided with all open orders (those where the drugs have not yet been received), which may be listed by invoice number (if available) or by office, vendor, number of doses, etc., although not limited thereto. Once the user identifies the invoice for the drug order in the system, the drug expiration date from the order may be entered into the system. It may be useful to track this so that the system can alert predetermined recipients when a drug in inventory is nearing expiration. The user may confirm that the initial drug order matches the packing slip and be prompted to enter additional information (e.g., delivery date, drug expiration date, drug lot number, etc.).

If the packing slip data from the received order does not match an invoice number for any open orders, the user may label the received order as a “non-scheduled delivery.” In this case, the system may provide the user the ability to supply relevant data, including packing slip data, drug type, number of doses, etc.

Tracking identifiers may be used to assist in tracking drug doses. For example, bar code labels may be printed by a printer 130 and affixed to each dose as they are received. The printed tracking information may also include the expiration date. Each dose may be scanned with a scanner 132 at particular times (e.g., inventory review, dose administration, new order receipt, etc.) for tracking purposes.

The system may provide inventory controller 116 to help manage the inventory of drug doses. In one embodiment, inventory adjustment may be used in cases including removing damaged or unusable doses from inventory, replacing doses removed for un-usability, removing purchased doses to be used for non-paying patients, replacing purchased doses previously used for non-paying patient, removing doses to be re-assigned to another location, replacing doses now re-assigned to another location, etc., although not limited thereto.

The display 118 may provide for drug dose assignment and un-assignment. For example, drug doses may be assigned to a patient when the dose is administered/dispensed. The user may be provided with a list of patients with appointments in the practice location chosen on the current day or from a list of all practice patients. If the selected patient is within minimum time since the last dose was dispensed to the patient, then a message may be displayed along the lines of “patient has received this drug X days ago.” If the patient is new to the practice the user may be given the option of adding the patient to the system “on the fly.” In this case, the user may be prompted to type the patient name and any other useful information such as the date and time of the treatment and the drug dose information. The user may be prompted to add the patient's diagnosis for comparison with insurance company rules entered by the user.

Once a dose has been assigned to a patient, the user may be prompted to scan the dose with a scanner 132. If the expiration date of the scanned dose is not at least equal to the oldest date in the inventory then a message such as the following may be displayed: “older expiration dates exist in the inventory, unassign and re-assign dose to use oldest inventory”. The available inventory may be decreased after the dose has been assigned. If the dose is for non-paying patient it may be classified as a “non-billable dose.” One objective of the system is to record each dose used and associate it with a patient. Information that the system may store includes dose information, invoice, patient information, date delivered, office delivered, etc.

It may also be possible to un-assign a dose using the display 118. If, for whatever reason, the dose is not administered to the patient the dose may be placed back into inventory. In this case the user may unassign the dose and be prompted to scan the dose. Once the dose is scanned the user may place the dose back into inventory and the total inventory count may be increased.

The inventory controller 116 may store the real-time total inventory count for each drug and each practice location, although not limited thereto. The first system user to log into the system each day (or at some other predetermined time) may be instructed to perform an inventory count to see if the physical inventory matches the inventory count stored in the system. If they do not match an error notice may be displayed including “count incorrect, click here if you wish to count again” or click “inventory count mismatch,” although not limited thereto. The system may also automatically lock the system (e.g., not provide any more inventory adjustments, etc.) and notify a manager (or some other predetermined recipient) of any mismatch, requiring the manager to log in and adjust the inventory to unlock the system. In this case, the manager may enter the number of doses currently in the physical inventory. The manager may also enter a comment explaining the inventory adjustment and the system may store the date, office, drug, number of doses plus or minus, manager information, and the manager comments regarding the event. This data may be used in reports, discussed further below. In addition, an alert (e.g., email, etc.) may be sent to predetermined recipients notifying them of the inventory issue.

Vendor payment functionality may allow the user to enter payment information against each order received. Most drug orders have payment terms of 60 days or greater. Therefore, it is possible that the practice may receive payment from insurance payers for doses administered to patients before the practice has paid the vendor for the doses. This results in “false profit.” The system may use vendor payment information in the calculation of false profit.

The system may provide reporter 110 that generates a variety of reports, discussed further below. For example, the system may provide a report for doses used, which may show all doses dispensed in a particular time period. This report may allow a user to select drug information, office information, etc., in order to filter and/or sort the report, although not limited thereto. It is to be appreciated that any number of filters and sorts can be used with the reports disclosed herein. Furthermore, any number of reports could be created using the system according to the present teachings. Disclosed herein are exemplary embodiments that are provided to aid in understanding the functionality of the present teachings that are not limited to any particular embodiment.

The system may provide a report for billing accuracy. For example, the report may report on the billing accuracy by drug and office location, although not limited thereto. The practice management system may be used to generate bills for drugs doses to the appropriate third party payer. In one embodiment, the inventory system tracks every dose assigned to a patient and confirms whether a properly configured bill has been entered in the PMS for each assigned doses. The system may attempt to match each patient and dose delivery date for that drug and check that the correct units were billed. If there is a match in name and date but the drug code does not match or the drug code matches but the wrong units were billed, the system may provide an alert and report that mismatch. The report may show drug information, patient information, procedure information, billing status (e.g., whether match found, etc.), drug billed, units billed, etc. If a bill is reported as absent or incorrectly configured by the inventory system and is subsequently added or corrected in the PMS, the error may no longer be displayed in the billing accuracy report.

Other reports may include, although not limited thereto, total payments to practice for drugs used (e.g., by invoice, invoice group, etc.), doses not used in particular timeframe, purchased doses, doses removed from inventory and not replaced, all assigned doses, false profit, profit in date range, inventory mismatch events, scheduled orders not received, current inventory, doses with bill but not assigned, vendor payment report, audit report, etc.

The system may provide a report on total payments to the practice for drugs used by invoice (e.g., by invoice, invoice group, date range, office, office group, etc.). The user may be provided with the ability to select drug information, office information, doses delivered by a particular doctor, date range, etc. Such a report may show each invoice, total payments to drug company due or paid for an invoice, the sum of total payments received for doses in the invoice, associated invoices, total payments received for associated invoices, etc. The report may show detailed information for each dose in the invoice as unassigned or assigned. For example, if a dose is assigned it may show the bar code (e.g., tracking information, etc.), patient information, the charge, the allowable amount, the total paid to date, etc.

The system may provide a report for total payments to a practice for drugs used by dose (e.g., doses delivered in date range, doses by office or office group, etc.). The report may provide the user the ability to select the drug information, office information, date range, etc. The report may show for a particular time period the drug information, bar code, patient information, date delivered, charge, the allowable amount, total amount paid, etc. Additionally, the report may show if the total paid is less than the allowable.

The system may provide a report for doses not used in a particular time period (e.g., last X days, by office or office group, etc.). The user may be able to select drug information, the time period, etc. In this way, it is possible to determine if any doses in current inventory have not been used in a particular number of days since receipt. The report may show drug information, dose information, office information, etc.

The system may provide a report for all purchased doses removed from inventory and not replaced. The user may be able to select drug information, office information, etc. The report may show information on the user who removed the dose, the reason for removal, dose number, associated invoice, etc.

The system may provide a report for false profit sum. The user may be able to select drug information, office information, etc. The report determines all doses for which the vendor has not yet been paid and for each of those doses determines the total receipts. If total receipts for a dose are less than the amount owed for the dose, then the total receipts are added to false profit sum. If the total receipts are greater than amount owed for the dose, then the amount owed is added to the false profit sum.

The system may provide a report showing sum of profits in a particular date range as defined below. The user may be able to select drug information, office information, date range, etc. The report determines each dose used and paid for. For each such dose the amount received minus the amount paid is determined and added to the profit in date range sum. For each dose used and not yet paid for, if the total receipts are greater than the amount owed (e.g., amount owed for invoice divided by number of doses in invoice), then the amount received minus the amount owed may be determined. If the amount received is less than the amount owed for the dose, then nothing is added to the false profit sum.

The system may provide a report for inventory mismatch events. The user may be able to select office information, date range, etc. The report may show all events where an inventory count entered by a user did not match the stored inventory count. The report may display drug information, date, office information, number of doses plus or minus, information for a user that reset the inventory count, the comments regarding event, etc.

The system may provide a report for orders not received. The user may be able to select drug information, office information, date range, etc. The report may show any orders placed in the date range that have not been received to date.

The system may provide a report for current inventory (e.g., by drug, by office, etc.). The user may be able to select drug information, office information, etc. The report may show total doses and, for each office, for example, dose information, bar code, invoice, expiration date, etc.

The system may provide a report for doses billed but not assigned. The user may be able to select drug information, office information, data range, etc. The report may show any doses that appear as billed in the PMS 104 but for which there is no associated dose assigned in the system. The report may show office information, dose information, bar code, drug information, date billed, etc.

The system may provide an audit report showing each time a dose was handled, which user handled the dose, and what action was taken. The system may provide a report that shows different aspects of vendor payments such as order number, vendor, type of payment (e.g., credit card, check, purchase order), amount, etc. Credit against a vendor may be added and such credit may be applied against an invoice.

As shown in FIG. 1, the user computer 106 may be in communication with one or more scanners 132 and one or more printers 130. These devices may be used to facilitate tracking of drug inventory. The user may be given the option of scanning a drug dose with a scanner (e.g., barcode scanner, quick response code scanner, or some similar device). In one embodiment, this functionality (or other functionality) may be provided by an application on a smart phone. For example, although not limited thereto, the smart phone's camera may be used to take a picture of the drug dose (e.g., bar code, etc.) and transmit it to the system. In addition, the user may scan a patient's identification bracelet (or some other identifier) in order to associate the patient in the system, although not limited thereto. In one embodiment, bar codes may be affixed to each drug dose whereby the dose may be scanned and tracked in the system at predetermined times. A printer 130 may be used to create bar codes for received drugs. It is to be appreciated that the present teachings are not limited to any particular tracking technology, and one skilled in the art would appreciated that printers and scanners using various technologies may be employed to assist in tracking drug doses according to the present teachings. For example, bar codes, quick response codes, radio frequency identification, etc. may be used, although not limited thereto.

The system may provide security controller 119 that may control access to system functionality through the use of access lists, privileges, or some other technology. It may be preferable to maintain Health Insurance Portability and Accountability Act (HIPPA) and Health Information Technology for Economic and Clinical Health Act protected health information (HiTech PHI) security compliance. In this case, practice groups may be kept separate, a password given to each user (which may not be user-definable), manager passwords may require changes (e.g., every 60 days), etc. A user of the system may be prompted to enter login credentials such as a username and password in order to assure they have the necessary privileges to perform any task.

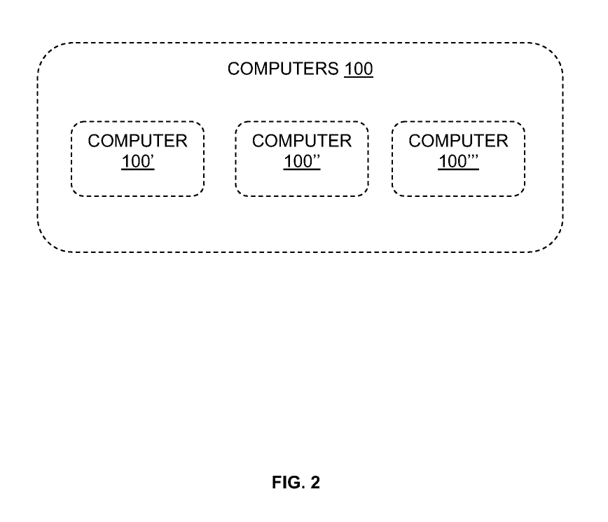

Referring now to FIG. 2, shown is a schematic diagram depicting the server computer 100 comprising multiple computers 100′, 100″, 100′″. It is appreciated that any number of processors and computers may be utilized to implement the present teachings, whether remote from each other and connected over a network, or local to each other, and the present teachings are limited to any particular embodiment disclosed herein.

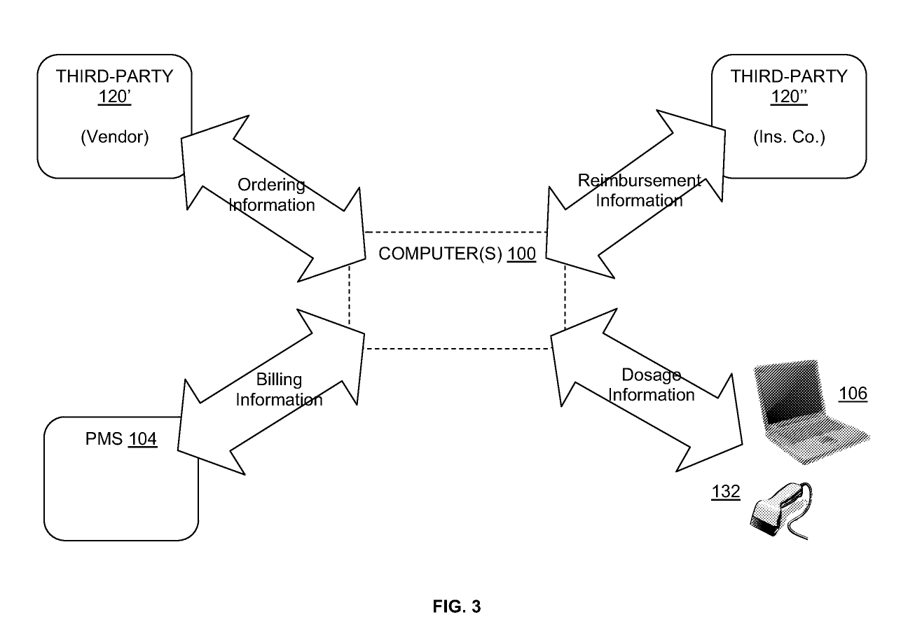

Referring now to FIG. 3, shown is a schematic diagram depicting data flow of the system of FIG. 1. In one exemplary use-case, the user of the system may associate a drug dose with a patient. This may be performed by scanning the patient bracelet and/or the drug dose with the scanner 132, although not limited thereto. In one alternative, the user computer 106 may be used. The dosage information may then be transmitted to the server computer 100. The server computer 100 may then receive billing information (e.g., insurance information, patient information, practice information, drug dosage information, payment information, etc.) from the PMS 104. The server computer 100 may then send a reimbursement request to the third-part insurance company 120″ using the patient's insurance information or via the PMS. In addition, the server computer 100 may determine that the inventory level for the particular drug is low and send an order for new drug doses to a third-party vendor 120′. It is to be appreciated that this is but one exemplary embodiment of the present teachings, which are not intended to be limited in this way.

While the present teachings have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

What is claimed is:

1. An inventory management system for a physician's office, comprising:
a database storing drug dosage inventory information;
a server computer in electronic communication with the database;
an interface that receives, over a network, patient information relating to a patient from a physician's office practice management system and stores it in the database;
an inventory controller that tracks an inventory of drug doses for the physician's office; and
a display that provides system access to a user computer over a network, including the association of a drug dose with the patient information in the database;
wherein the inventory controller determines an inventory mismatch between a physical inventory of drug doses and an inventory count in the database.

2. The system of claim 1 wherein the physician's office is engaged in the field of retinal disorders.

3. The system of claim 1 further comprising a reimbursement controller for sending a reimbursement request to an insurance company for a drug dose administered to the patient.

4. The system of claim 3, wherein the reimbursement request is sent via the physician's office practice management system.

5. The system of claim 1 wherein the inventory controller creates an order invoice for an order of drug doses from a vendor.

6. The system of claim 5 wherein the inventory controller transmits the order invoice to the vendor.

7. The system of claim 1 wherein the system alerts one or more predetermined recipients when a drug dose inventory for a drug falls below a predetermined threshold.

8. The system of claim 1 wherein the system alerts one or more predetermined recipients of one or more of the following events: a drug dose nears expiration, the patient has been associated with a drug dose within a minimum time for administering the drug dose, and a drug dose exists in inventory that is older than a drug dose that has been associated with the patient.

9. The system of claim 1 wherein when the system identifies an inventory mismatch the system locks certain functionality until a predetermined user adjusts the inventory count.

10. The system of claim 1 wherein the server computer comprises a plurality of computers in electronic communication with each other.

11. The system of claim 1 wherein the display comprises a web page.

12. The system of claim 1 wherein the user computer accesses the system over the Internet.

13. The system of claim 1 wherein the user computer is a mobile device.

14. The system of claim 1 wherein the inventory controller determines if there is a discrepancy between a drug dose order and a received drug dose shipment.

15. The system of claim 1 wherein a patient diagnosis is compared with an insurance company payment policy and warns the user of a reimbursement issue if the drug is administered to a patient with the patient diagnosis.

16. The system of claim 1 further comprising a reporter that provides a plurality of reports to users of the system relating to the drug dosage inventory information.

17. The system of claim 1 further comprising a scanner, wherein the scanner scans a drug dose for tracking it in the system.

18. The system of claim 1 further comprising a bar code printer and bar code scanner, wherein the bar code printer prints a bar code for a drug dose and the bar code scanner scans the drug dose.

19. A method of managing inventory for a physician's office, comprising the steps of:
providing a database storing drug dosage inventory information;
providing a server computer in electronic communication with the database;
receiving, over a network, patient information relating to a patient from a physician's office practice management system and storing it in the database;
tracking, on a computer, an inventory of drug doses for the physician's office; and
providing access to the server computer to a user computer over a network, including the association of a drug dose with the patient information in the database;
wherein the inventory controller determines an inventory mismatch between a physical inventory of drug doses and an inventory count in the database.

20. The method of claim 19 wherein the physician's office is engaged in the field of retinal disorders.

21. The method of claim 19 wherein when the inventory controller identifies an inventory mismatch certain functionality is locked until a predetermined user adjusts the inventory count.

22. The method of claim 19 wherein the step of providing access includes providing a web page.

23. The method of claim 19 wherein the user computer accesses the server computer over the Internet.

24. The method of claim 19 further comprising the step of determining whether a properly configured bill has been entered in the physician's office practice management system for an assigned drug dose.

25. The method of claim 19 wherein the physician's office is engaged in the field of general practice.
